(19) United States
(12) Patent Application Publication
(10) Pub. No.: US 2008/0108665 A1
(43) Pub. Date: May 8, 2008

(54) Use of N-substituted sulfoximines for control of invertebrate pests

(76) Inventors: Jim X. Huang, Carmel, IN (US); Yuanming Zhu, Carmel, IN (US); Richard B. Rogers, Mobile, AL (US); Michael R. Loso, Carmel, IN (US); Robert L. Hill, Carmel, IN (US); James D. Thomas, Fishers, IN (US); Thomas Meade, Zionsville, IN (US); James M. Gifford, Lebanon, IN (US); Joseph J. DeMark, Westfield, IN (US); Benjamin M. Nugent, Brownsburg, IN (US)

Correspondence Address: DOW AGROSCIENCES LLC, 9330 ZIONSVILLE RD, INDIANAPOLIS, IN, 46268, US

(21) Appl. No.: 11/704,397

(22) Filed: Feb. 9, 2007

Related U.S. Application Data
(60) Provisional application No. 60/857,709, filed on Nov. 8, 2006.

(51) Int. Cl. A01N 43/40; C07D 213/57; C07D 411/02

(52) U.S. Cl. 514/336; 514/357; 546/280.4; 546/330

(57) ABSTRACT

Methods to control certain invertebrates including insects in agricultural, urban, animal health, and industrial systems by directly or systemically applying to a locus where control is desired an effective amount of a compound of N-substituted sulfoximines.

This application claims the benefit of U.S. Provisional Application Ser. No. 60/857,709 filed on Nov. 8, 2006.

### BACKGROUND OF THE INVENTION

The present invention concerns using N-substituted sulfoximines to control invertebrate pests. This invention also includes agricultural and/or nonagricultural applications suitable for the compounds, compositions containing the compounds, and methods of controlling invertebrate pest using the compounds.

The development of resistance to some insecticides, including DDT, the carbamates, the organophosphates and the pyrethroids, is well known. The introduction of neonicotinoid insecticides has provided growers with invaluable new tools for managing some of the world's most destructive crop pests, primarily those of the orders Homoptera and Coleoptera, including species with a long history of resistance to earlier-used products. Imidacloprid was the first major active ingredient of the neonicotinoid class to reach the market. Research on molecules with a similar structure containing the 6-chloro-3-pyridylmethyl moiety led to acetamiprid, nitenpyram and thiacloprid. The substitution of the chloropyridinyl moiety by a chlorothiazolyl group resulted in a second subgroup of neonicotinoid insecticides including clothianidin and thiamethoxam. Although these neonicotinoids have proved relatively resilient to the development of resistance, the susceptibility of Myzus persicae may vary up to 20-fold between populations (Foster et al., 2003 Pest Manag Sci. 59: 166-173; Nauen and Denholm, 2005 Arch Insect Biochem Physiol. 58:200-215). Stronger resistance has been confirmed in some populations of sweet potato whitefly, Bemisia tabaci. During the late 1990s, resistant species increased in potency with more recently-collected strains of this whitefly—exhibiting more than 100-fold resistance to imidacloprid, and comparable levels of resistance to thiamethoxam and acetamiprid (Elbert and Nauen, 2000 Pest Manag Sci. 56: 60-64; Rauch and Nauen, 2003 Arch Insects Biochem Physiol. 54: 165-176; Gorman et al., 2003 Proc BCPC Intl Cong: Crop Science & Technology. 2: 783-788). Resistance to imidacloprid has also been found in another key target species, Colorado potato beetle (Leptinotarsa decemlineata, CPB). Currently there are 42 active ingredients across several classes, including organophosphates, carbamates and pyrethroids, with reported resistance in CPB (Whalon et al., 2006 MSU database of pesticide resistance http://www.cips.msu.edu/resistance/rmdb). Imidacloprid was the only registered neonicotinoid insecticide for CPB control in the potato-growing regions of USA until 2002 when thiamethoxam (and later other neonicotinoids) were introduced to control CPB. High levels of resistance to imidacloprid were found in adult CPB from Long Island, N.Y., and cross-resistance was seen to all the commercial neonicotinoids tested (Mota-Sanchez et al., 2006 Pest Manag Sci. 62: 30-37). Therefore a need exists for new pesticides, and particularly for compounds that have new or atypical modes of action, or compounds that possess sufficient potency and special attributes allowing effective control of pests in agricultural, urban and/or animal health systems through versatile approaches such as spray, seed treatment, irrigation, bait and the like.

### SUMMARY OF THE INVENTION

This invention concerns controlling invertebrate pests in agricultural, urban, animal health, and industrial systems by directly or systemically applying to a locus where control is desired an effective amount of a compound of formula (I)

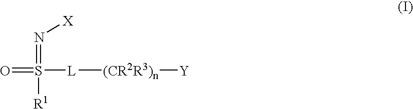

wherein

X represents NO₂, CN or COOR⁴;

L represents a single bond or —CH—(CH₂)ₘ wherein m is an integer from 1-3 in cases where R¹, S and L taken together represent a 4-, 5- or 6-membered ring;

R¹ represents methyl, ethyl, or —CH₂— in cases where R¹, S and L taken together represent a 4-, 5- or 6-membered ring;

R² and R³ independently represent hydrogen, methyl, ethyl, fluoro, chloro or bromo;

n is an integer from 0-3;

Y represents 6-halopyridin-3-yl, 6-(C₁-C₄)alkylpyridin-3-yl, 6-(C₁-C₄)alkoxypyridin-3-yl, 2-chlorothiazol-4-yl, 2-chlorothiazol-5-yl, or 3-chloroisoxazol-5-yl; and

R⁴ represents C₁-C₃ alkyl.

Preferred compounds of formula (I) include the following classes:

(1) Compounds of formula (I) wherein X is NO₂ or CN, most preferably CN.

(2) Compounds of formula (I) wherein R¹, S and L taken together form a standard 5-membered ring, n=1, and Y represents 6-chloropyridin-3-yl, i.e., having the structure

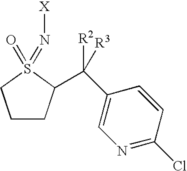

wherein

X represents CN; and

R² and R³ independently represent hydrogen or methyl.

(3) Compounds of formula (I) wherein R¹, S and L taken together form a standard 5-membered ring and n=0, i.e., having the structure

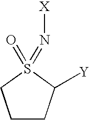

wherein

X represents CN; and

Y represents 6-halopyridin-3-yl.

(4) Compounds of formula (I) wherein R¹ represents CH₃, L represents a single bond and Y represents 6-chloropyridin-3-yl, i.e., having the structure

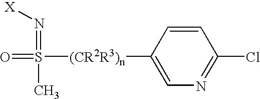

wherein

X represents CN;

R² and R³ independently represent hydrogen or methyl; and

n=1-3.

It will be appreciated by those skilled in the art that the most preferred compounds are generally those which are comprised of combinations of the above preferred classes.

The invention provides specific uses of compounds of formula (I) which will be described in detail hereinafter.

### DETAILED DESCRIPTION OF THE INVENTION

Throughout this document, all temperatures are given in degrees Celsius, and all percentages are weight percentages unless otherwise stated.

Unless specifically limited otherwise, the term alkyl (including derivative terms such as alkoxy), as used herein, include straight chain, branched chain, and cyclic groups. Thus, typical alkyl groups are methyl, ethyl, 1-methylethyl, propyl, 1,1-dimethylethyl, and cyclopropyl. The term halogen includes fluorine, chlorine, bromine, and iodine.

The compounds of this invention can exist as one or more stereoisomers. The various stereoisomers include geometric isomers, diastereomers and enantiomers. Thus the compounds of the present invention include racemic mixtures, individual stereoisomers and optically active mixtures. It will be appreciated by those skilled in the art that one stereoisomer may be more active than the others. Individual stereoisomers and optically active mixtures may be obtained by selective synthetic procedures, by conventional synthetic procedures using resolved starting materials or by conventional resolution procedures.

Methods for the preparation of sulfoximines have been previously disclosed in US Patent Publication 2005/0228027, whose teachings are incorporated herein.

### EXAMPLES

### Examples I-III

### Preparation of N-Substituted Sulfoximines

Sulfoximines I-III are prepared by methods previously disclosed in US Patent Publication 2005/0228027.

### Example I

### Preparation of [(6-Chloropyridin-3-yl)methyl](methyl)oxido-λ⁴-sulfanylidenecyanamide (1)

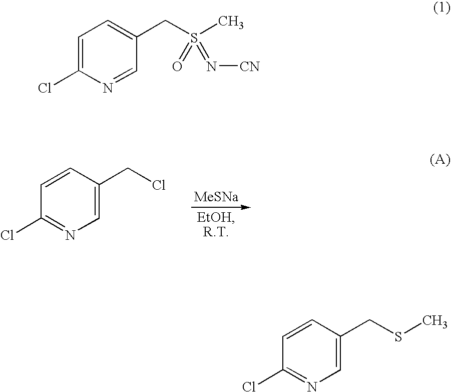

A solution of 5-chloromethyl-2-chloropyridine (8.1 g, 50 mmol) in ethanol (50 mL) was added to a suspension of sodium thiomethoxide solid (4.2 g, 60 mmol) in 100 mL ethanol under stirring. An exothermic reaction was observed during the addition and the mixture was then stirred at room temperature overnight.

The solvent ethanol was removed under reduced pressure and the residue was re-dissolved in ether-EtOAc solvent and mixed with brine. The two phases were separated and the organic layer was dried over anhydrous Na₂SO₄, filtered, concentrated and purified by briefly passing through a silica gel plug by elution with 40% EtOAc in hexane to give 8.14 g of 2-chloro-5-[(methylthio)methyl]-pyridine as a colorless oil in 94% yield. The product was analytically pure and directly used for the next step reaction. ¹H NMR (300 MHz, CDCl₃) δ 8.28 (dd, 1H), 7.65 (dd, 1H), 7.30 (d, 1H), 3.63 (s, 2H), 2.00 (s, 3H).

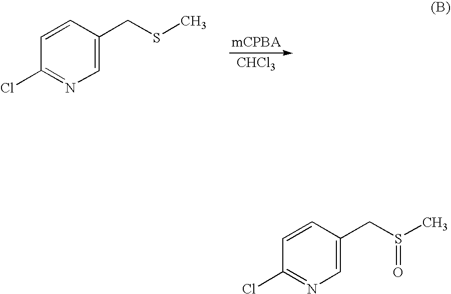

To a solution of 2-chloro-5-[(methylthio)methyl]pyridine (7.60 g, 40 mmol) in chloroform (100 mL) cooled in ice-water bath was added a solution of 70-75% m-chloroperbenzoic acid (mCPBA, 10.51 g, ca 44 mmol) in chloroform (110 mL) over a period of 1.5 h under stirring. The stirring was continued for another 1 h at 0° C. Methanol (12 mL) was added to the mixture, which was then bubbled with ammonia gas to precipitate the benzoic acid and the extra mCPBA, if any. TLC showed there was small amount of unreacted starting material in the reaction mixture. Most of the ammonium salt was removed by filtration. The filtrate was concentrated before it was loaded onto a Et₃N-treated silica gel plug, and was eluted with 10% MeOH in CH₂Cl₂ to give 7.6 g of the 2-chloro-5-[(methylsulfinyl)methyl]pyridine (92% yield) as a colorless oil that turned into a white solid upon drying in vacuo, m.p. 72-74° C. LC-MS (ELSD): mass calcd for C₇H₈ClNOS [M+H]⁺190.67. Found: 190.21.

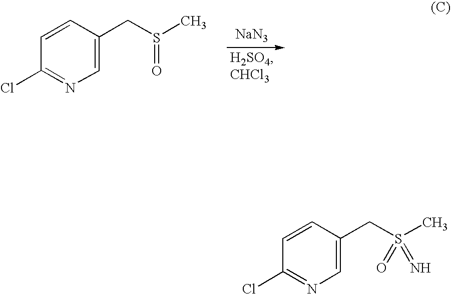

To a stirred mixture of 2-chloro-5-[(methylsulfinyl)methyl]pyridine (7.34 g, 39 mmol) and sodium azide (4.04 g, 62 mmol) in chloroform (80 mL) cooled to 0° C. was slowly added sulfuric acid at a rate such that the temperature did not rise above 8° C. After the addition was over, the ice-water bath was removed and the mixture was heated at 55° C. for 2.5 h.

The solvent was decanted into a separation funnel and the sticky residue was stirred and dissolved in water. After a few minutes, the aqueous mixture was made basic to pH 8 by slowly adding solid Na₂CO₃ and then saturated with solid NaCl. The extra salt was removed by filtration (a portion of the filtrate was sucked into trap and was not recovered) and the filtrate was extracted with CH₂Cl₂ three times. The combined organic layer was dried over Na₂SO₄, filtered, concentrated and the residue was triturated in CH₂Cl₂-ether (1:10, v/v) solvent. The white solid was filtered, washed with CH₂Cl₂-ether (1:10, v/v) and dried to give 2.70 g 2-chloro-5-[(methylsulfonimidoyl)methyl]pyridine (34%) as a white solid, m.p. 134.5-136° C. LC-MS (ELSD): mass calcd for C₇H₉ClN₂OS [M+H]⁺203.67. Found: 203.22.

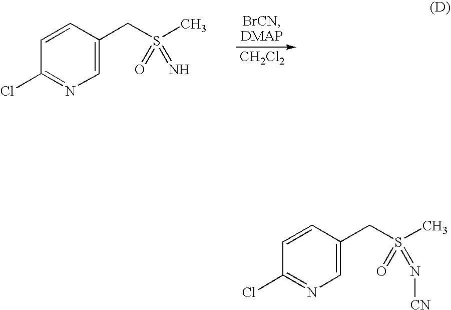

To a stirred mixture of 2-chloro-5-[(methylsulfonimidoyl)methyl]pyridine (2.04 g, 10 mmol) and 4-dimethylaminopyridine (DMAP, 1.22 g, 10 mmol) in CH₂Cl₂ (15 mL) was added cyanogen bromide (5.0 mL, 15 mmol). An exothermic reaction was observed. The resulting solution was stirred at room temperature for 2 h and quenched with 2 N aqueous HCl solution. After separation of the two phases, the aqueous layer was extracted with CH₂Cl₂ three times. The combined organic layer was dried over Na₂SO₄, filtered, concentrated, and purified on silica gel using 5% MeOH in CH₂Cl₂ as eluent to give 1.51 g of [(6-chloropyridin-3-yl)methyl](methyl)oxido-λ⁴-sulfanylidenecyanamide (1) in 66% yield as a greenish oil which turned into a greenish solid while being dried under vacuum, m.p. 115-117° C. ¹H NMR (300 MHz, CDCl₃+CD₃OD) δ 8.49 (d, 1H), 7.96 (dd, 1H), 7.55 (d, 1H), 4.98 (s, 2H), 3.40 (s, 3H). ¹³C NMR (300 MHz, CDCl₃+CD₃OD) δ 153.6, 152.7, 143.2, 125.8, 123.1, 113.2, 57.86, 39.97. LC-MS (ELSD): mass calcd for C₈H₈ClN₃OS [M−H]⁺ 228.68. Found. 228.19

### Example II

### Preparation of [1-(6-chloropyridin-3-yl)ethyl](methyl)oxido-λ⁴-sulfanylidenecyanamide (2)

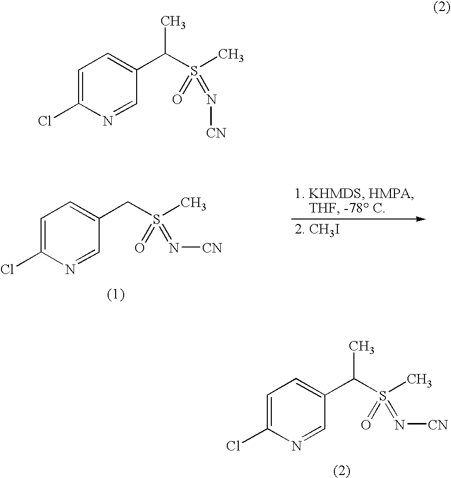

To a solution of N-cyanosulfoximine (1) (0.34 g, 1.5 mmol) and hexamethyl phosphoramide (HMPA) (0.14 mL, 0.8 mmol) in 15 mL anhydrous tetrahydrofuran (THF) was added dropwise a solution of 0.5 M potassium bis(trimethylsilyl)amide (KHMDS) in toluene (3.6 mL, 1.8 mmol) at −78° C. After 45 min, iodomethane (0.11 mL, 1.8 mmol) was added in one portion via a syringe. Ten minutes later, the temperature was allowed to rise to 0° C. After stirring for 1.5 h., the reaction was quenched with saturated aqueous NH₄Cl, diluted with brine and extracted with CH₂Cl₂ three times. The combined organic layer was dried over Na₂SO₄, filtered and concentrated. The residue was first purified on silica gel twice, first time eluted with 2% MeOH in CH₂Cl₂ (v/v) and the second time with 9% acetone in CH₂Cl₂ (v/v) to give 0.217 g of mono methylated N-cyano sulfoximine (2) in 60% yield ([M−H]⁺=242, 244) as a mixture of disastereomers. ¹H NMR (300 MHz, CDCl₃) δ 8.47 (m, 1H), 7.88 (m, 1H), 7.48 (m, 1H), 4.63 (q, 1H), 3.11 & 3.10 (m, 3H), 1.98 (m, 3H). ¹³C NMR (300 MHz, d₆-DMSO) δ 151.46 &151.22, 140.80 &140.64, 127.55 &127.31, 124.60 &124.58, 112.27 & 111.96, 62.31 & 62.25, 37.68 &37.47m 13.34 &13.29. LC-MS (ELSD): mass calcd for C₉H₁₀ClN₃OS [M−H]⁺ 243.72. Found 242.21.

### Example III

### Preparation of 2-[(6-chloropyridin-3-yl)methyl]-1-oxidotetrahydro-1H-1-λ⁴-thien-1-ylidenecyanamide (3)

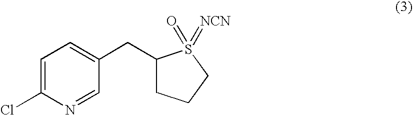

The starting material 1-oxidotetrahydro-1H-1-λ⁴-thien-1-ylidenecyanamide (4) was prepared from tetrahydrothiophene-1-oxide by a two-step procedure as described in Example I, steps (C) and (D): imination of the sulfoxides with sodium azide and N-cyanation of the resulting sulfoximine with cyanogen bromide. ¹³C NMR (CDCl₃): 112.3, 52.9.

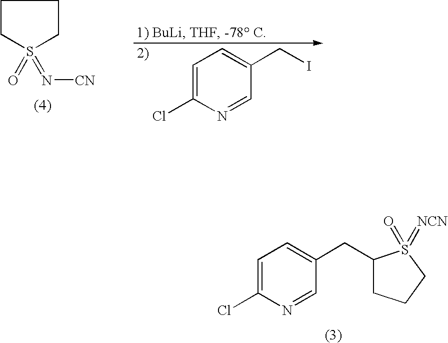

To a solution of N-cyano sulfoximine (0.164 g, 1.0 mmol) in 8 mL THF was added 2.5 M BuLi in hexane (0.44 mL, 1.1 mmol) at −78° C. After 1 h, a suspension of 2-chloro-5-iodoomethylpyridine in 3 mL THF was added in one portion via a syringe. After 30 min, the mixture was stirred at room temperature for 3 hrs. The reaction was quenched with ammonium chloride and worked up. The crude product was first loaded onto a silica gel column eluted with 5% MeOH in CH₂Cl₂, followed by reverse-phase preparative HPLC to give 0.106 g of desired product 3 in 34% yield as a mixture of disastereomers. ¹H NMR (300 MHz, CDCl₃) δ 8.31 (m, 1H), 7.64 (m, 1H), 7.34 (dd, 1H), 3.24-3.81 (m, 4H), 2.89-3.40 (m, 1H), 1.91-2.52 (m, 4H). LC-MS (ELSD): mass calcd for C₁₁H₁₄ClNOS [M−H]⁺ 268.74. Found 268.27.

### Example IV-X

### Insecticidal Testings

The compounds identified in the foregoing examples were tested against cotton aphid, green peach aphid, brown planthopper and/or green leafhopper through root uptake and seed treatment, against termites through contact and ingestion, against cat flea and brown dog tick through contact using procedures described hereinafter.

### Example IV

### Insecticidal Test for Green Peach Aphid (Myzus persicae) in Root Uptake Assay

These assays were designed to evaluate the possibility of using the compounds identified in the foregoing examples for applications such as nursing trays, transplanting water and/or irrigation. Systemic activity against green peach aphid was evaluated in a root uptake assay. Bell peppers (Capsicum annum var. California Wonder) were used as test plants seeded and grown in rock wool plugs. Plants were grown to expanding 1^{st} true leaf stage. The rock wool plugs containing individual plants were placed in 1-ounce cups and surrounded with white clean sand. Five plants were used for each treatment. A volume of 5 mL of the test solutions was applied to each cup (each plant). After insecticide application, the seedlings were infested with green peach aphids and held in a growth chamber (25° C., 50% RH, 16 hr light: 8 hr dark). Number of live aphids on each plant was counted at 3 days after infestation. Calculations for % Control were based on a corrected basis compared to the populations on the reference plants.

Corrected % Control=100*(X−Y)/X
where X=No. of live aphids on reference plants
   Y=No. of live aphids on treated plants
      Assay 1: Compound (3) was tested at a dose range of 5-500 μg/plant. A stock solution of 1000 ppm (1 mg/mL) was made by dissolving technical test compound in acetone:ethanol (90:10). The highest test concentration (100 ppm, 500 μg/5 ml) was prepared by diluting 2.5 mL stock solution with 22.5 mL DI water. The lower test concentrations (25 mL) were similarly prepared by diluting aliquots of the stock solution with DI water. Reference plants received DI water only. The Corrected % Control values from the test rates are given in Table 1.

TABLE 1
Systemic activity against green peach aphid on pepper.

| Comp # | Dose, μg/plant | % Control, root uptake systemic |
|---|---|---|
| 3 | 5 | 10 |
| | 50 | 88 |
| | 500 | 100 |

Assay 2: Compound (2) was tested at a dose range of 0.08-50 μg/plant. A stock solution of 1000 ppm was made by dissolving 2 mg of technical test compound in 2 mL acetone. The highest test concentration (10 ppm, 50 μg/5 ml) was prepared by diluting 0.32 mL stock solution with 1.6 mL acetone and 30.08 mL DI water, containing 6% acetone. Lower test concentrations were prepared by sequentially diluting 6.5 mL higher concentration (start from the 10 ppm test solution) with 26.0 ml acetone: DI water (6:94). The solvent/diluent system (6% acetone) was used as reference. The Corrected % Control values from the lower test rates are given in Table 2.

TABLE 2
Systemic activity against green peach aphid on pepper.

| Comp # | Dose, μg/plant | % Control, root uptake systemic |
|---|---|---|
| 2 | 0.08 | 41 |
| | 0.4 | 48 |
| | 2 | 95 |
| | 10 | 99 |

### Example V

### Insecticidal Test for Brown Planthopper (Nilaparvata lugens) and Green Leafhopper (Nephotettix sp.) in Root Uptake Assays

These assays were designed to evaluate the possibility of using compounds of the invention for applications such as nursing trays, transplanting water and/or irrigation. Root-uptake systemic assays were performed on both brown planthopper and green leafhopper. Four-week-old rice seedlings were submerged in 3-cm depth of water in the bottom portion (high 5 cm, diameter 3 cm) of a 2-part glass cylinder (high 18 cm, diameter 3 cm). A metal screen was used to hold the seedlings within the bottom portion. Scotch tape was used to bind the two portions of the cylinder after setting up the seedlings. A metal cap was used to cover the cylinder. There were 4 cylinders for each treatment. The test compound was dissolved in acetone to make a 10,000 ppm stock solution which was incorporated at final test concentrations of 5, 2, 1, 0.5, 0.25 ppm in the water in which rice seedlings were submerged. Five laboratory-reared 3^{rd} instar nymphs of brown planthopper or green leafhopper were introduced into each cylinder 3 hr after insecticide application. The treated test units were kept in a growth chamber with conditions set as followings: Temperature 28±0.5° C.; Relative humidity 70±0.5%; Photoperiod 14 hr light: 8 hr dark. Mortality of hoppers was observed at 2 and 6 days after infestation. The corrected % Control values relative to mortality in solvent reference from the 6-day observation are given in Table 3.

TABLE 3
Systemic activity against hoppers on rice.

| | % Control, root uptake systemic | | |
|---|---|---|---|
| | Conc., | Brown | Green |
| Comp # | ppm | planthopper | leafhopper |
| 2 | 0.25 | 55 | 45 |
| | 0.50 | 80 | 65 |
| | 1.00 | 100 | 100 |
| | 2.00 | 100 | 100 |
| | 5.00 | 100 | 100 |

### Example VI

### Insecticidal Test for Cotton Aphid (Aphis gossypii) in Seed Coating Assay

Selected compounds of the invention were tested in assays designed to evaluate its systemic activity for control of cotton aphid through seed-coating prior to planting. The crop used in these assays was a hybrid squash (var. Pic-N-Pic). Test compound was formulated in a 10% SC formulation. Ten squash seeds were used for each treatment. Seeds were placed on waxed paper and a pipette was used to apply the original or diluted formulations to each seed. The air-dried, treated seeds were individually planted into 3 inch pots containing metro mix. The pots were placed on a California cart and moved into the greenhouse for sub watering only. At the specified number of days after planting, the plants were infested with approximately 40 wingless aphids. The infested plants were kept in an environmental holding room (23° C., 40% RH, 16 hr light: 8 hr dark) for three days before the number of live aphids was counted under a microscope. Calculations for % Control were based on a corrected basis compared to the populations on the reference plants germinated from seeds treated with the formulation blank.

Corrected % Control=100*(X−Y)/X
where X=No. of live aphids on reference plants
   Y=No. of live aphids on treated plants
      Assay 1. Compounds (1) and (2) were tested at 3 mg/seed. The original 10% SC formulation was used. One half (15 ul) of the sample was spread onto one side of a seed. Once dried (approximately 1 hr), the seed was flipped over and the 2nd half of sample was spread over the other side. Fourteen and 25 days after planting, aphid infestations were conducted on the 1^{st} and 2^{nd} true leaves, respectively. The Corrected % Control values from the assay are given in Table 4.

TABLE 4
Systemic activity against cotton aphid through squash seed treatment.

% Control, seed coating systemic

| | | 1^{st} leaf, 14 days | 2^{nd} leaf, 25 days |
|---|---|---|---|
| Comp # | Dose, mg/seed | after planting | after planting |
| 1 | 3 | 100 | 99.6 |
| 2 | 3 | 100 | 99.8 |

Assay 2. Compound (2) was tested at 0.1 and 1 mg/seed. For the 1 mg/seed treatment, the original 10% formulation was used. For the 0.1 mg/seed treatment, the 10% formulation were diluted 10× with DI water before application. One half (5 ul) of the sample was spread onto one side of a seed. Once dried (approximately 1 hr), the seed was flipped over and the 2nd half of sample was spread over the other side. Thirteen days after planting, the 1st leaf was infested. The Corrected % Control values from the assay are given in Table 5.

TABLE 5
Systemic activity against cotton aphid through squash seed treatment.

| Comp # | Dose, mg/seed | % Control, seed coating systemic |
|---|---|---|
| 2 | 0.1 | 36.5 |
| | 1 | 99.8 |

### Example VII

### Insecticidal Test for Bird Cherry-Oat Aphid (Rhopalosiphum padi) in Seed Coating Assay

Twenty spring wheat (Triticum aestivum var. Yuma) seeds were placed on a waxed paper. A pipette was used to apply a 10% SC formulation of the test compound to each seed. Two application of 6.7 μl each were applied to a seed, waiting until the seed was air-dried between applications. The total amount of active ingredient applied to each seed was 1.34 mg. All coated seeds are planted into 3 inch pots containing greenhouse metro mix, with 4 seeds used in each pot. An appropriate number of untreated seeds was also planted for control. The pots are placed on a California cart and moved into a greenhouse for initial and subsequent sub-watering. When the plants had germinated and grown to the 1-2 leaf unfurled stage, they were infested with bird cherry-oat aphid. The infested plants were held in an environmental chamber (23° C., 40% RH, 16 hr light: 8 hr dark) for 4 days before an assessment of aphid populations was conducted. Calculations for % Control are based on a corrected basis compared to the populations on the untreated controls.

Corrected % Control=100*(X−Y)/X
where X=No. of live aphids on untreated plants
   Y=No. of live aphids on treated plants

### The Corrected % Control values from assays are given in Table 6.

TABLE 6
Systemic activity against bird cherry-oat aphid through wheat seed
treatment.

| Comp # | Dose, mg/seed | % Control, seed coating systemic |
|---|---|---|
| 2 | 1.34 | 100 |

### Example VIII

### Insecticidal Test for Eastern Subterranean Termite (Reticulitermes flavipes) in Filter Paper Assay

Activity of Compound (2) was evaluated for its activity on Eastern subterranean termite. Technical material of the test compound was formulated in acetone on a wt/wt basis to deliver 1000, 500, 200, 50, 12.5, 3.12 and 0.78 ppm to 42.5 mm Whatman No. 1 filter papers per 200 ul of pipetted solution. Each test concentration was applied to six filter papers (6 reps). Six acetone-only control units and six DI water-only control units were also prepared. The filter papers were dried overnight in the fume hood before they were placed into 60×15 mm Fisher Brand plastic Petri dishes. A volume of 200 ul DI water was pipetted onto each Filter paper at the time of test set-up, just prior to infesting with termites. Ten worker termites were added to each Petri dish and covered. The infested Petri dishes were put in the laboratory Conviron at 28° C. and 60% RH. Termite mortality was recorded at 1, 2, 4, 7, and 10 days after infestation (DAI). Throughout the duration of the test, an average of 150 ul of DI water was added daily to the filter papers to retain moisture. Results are presented in Table 7.

TABLE 7
Activity against termite.

| | | % Termite mortality | | | | |
|---|---|---|---|---|---|---|
| Comp # | Conc., ppm | 1 DAI | 2 DAI | 4 DAI | 7 DAI | 10 DAI |
| 2 | 0.78 | 0 | 2 | 2 | 7 | 10 |
| | 3.13 | 0 | 3 | 43 | 60 | 60 |
| | 12.5 | 15 | 32 | 60 | 63 | 63 |
| | 50 | 15 | 27 | 67 | 92 | 93 |
| | 200 | 37 | 58 | 87 | 97 | 100 |
| | 500 | 38 | 60 | 93 | 100 | 100 |
| | 1000 | 47 | 68 | 92 | 100 | 100 |
| Acetone | — | 0 | 0 | 0 | 2 | 3 |
| Control | | | | | | |
| DI Water | — | 7 | 7 | 7 | 7 | 7 |
| Control | | | | | | |

### Example IX

### Insecticidal Test for Cat Flea (Ctenocephalides felis) in Filter Wool Assay

Compound (2) was evaluated in a dose response series to establish the range of activity of the test compound. Technical material was dissolved in acetone and diluted with the same solvent to obtain the test concentrations. Bioassays were conducted by treating polyester aquarium filter wool with 1.0 ml of the test solution, thoroughly saturating the substrate and allowing it to dry for at least 1 hour. The dry filter wool was then placed into 10 cm plastic Petri dishes and covered with the lid. Each treatment was replicated 5 times. Approximately 15 unfed cat flea adults were placed into each replicate of each dosage being evaluated. Mortality was assessed at 2, 8, 24 and 48 hours after introduction of the fleas into the test system. The mean percent mortality for each dosage group and time interval was determined and results from the 48-hour observation are presented in Table 8.

TABLE 8
Mean percent mortality of cat fleas after 48-hour exposure.

| Comp # | Conc., ppm | % Flea mortality |
|---|---|---|
| 2 | 0.5 | 52.4 |
| | 5.0 | 74.4 |
| | 50.0 | 76.6 |
| | 500.0 | 83.8 |
| | 5,000.0 | 100 |

### Example X

### Insecticidal Test for Brown Dog Tick (Rhipicephalus sanguineus) in Glass Plate Assay

Compound (2) was evaluated in a dose response series to establish the range of activity of the test compound. Technical material was dissolved in acetone and diluted with the same solvent to obtain the test concentrations. Tick bioassays were conducted by applying 1.0 ml of the test substance to clean dry glass plates confined by 10 cm grease pencil circles drawn on the plates and spread evenly with an acid brush. The plates were allowed to dry for at least 1 hour before adult ticks were confined to the treated substrate using 10 cm Petri dish lids. Each treatment was replicated 5 times. Approximately 5 adult ticks were placed into each replicate. Mortality was assessed at 2, 8, 24 and 48 hours after introduction of the ticks into the test system. The mean percent mortality for each dosage group and time interval was determined and results from the 48-hour observation are presented in Table 9.

TABLE 9
Mean percent mortality of brown dog ticks after 48-hour exposures.

| Comp # | Conc., ppm | % Tick mortality |
|---|---|---|
| 2 | 5.0 | 52.0 |
| | 50.0 | 56.0 |
| | 500.0 | 84.0 |

### Insecticide Utility

The compounds of the invention are useful for the control of invertebrates including insects. Therefore, the present invention also is directed to a method for inhibiting an insect which comprises applying an insect-inhibiting amount of a compound of formula (I) to a locus of the insect, to the area to be protected, or directly on the insect to be controlled. The compounds of the invention may also be used to control other invertebrate pests such as mites and nematodes.

The “locus” of insects or other pests is a term used herein to refer to the environment in which the insects or other pests live or where their eggs are present, including the air surrounding them, the food they eat, or objects which they contact. For example, insects which eat, damage or contact edible, commodity, ornamental, turf or pasture plants can be controlled by applying the active compounds to the seed of the plant before planting, to the seedling, or cutting which is planted, the leaves, stems, fruits, grain, and/or roots, or to the soil or other growth medium before or after the crop is planted. Protection of these plants against virus, fungus or bacterium diseases may also be achieved indirectly through controlling sap-feeding pests such as whitefly, plant hopper, aphid and spider mite. Such plants include those which are bred through conventional approaches and which are genetically modified using modern biotechnology to gain insect-resistant, herbicide-resistant, nutrition-enhancement, and/or any other beneficial traits.

It is contemplated that the compounds might also be useful to protect textiles, paper, stored grain, seeds and other foodstuffs, houses and other buildings which may be occupied by humans and/or companion, farm, ranch, zoo, or other animals, by applying an active compound to or near such objects. Domesticated animals, buildings or human beings might be protected with the compounds by controlling invertebrate and/or nematode pests that are parasitic or are capable of transmitting infectious diseases. Such pests include, for example, chiggers, ticks, lice, mosquitoes, flies, fleas and heartworms. Nonagronomic applications also include invertebrate pest control in forests, in yards, along road sides and railroad right of way.

The term “inhibiting an insect” refers to a decrease in the numbers of living insects, or a decrease in the number of viable insect eggs. The extent of reduction accomplished by a compound depends, of course, upon the application rate of the compound, the particular compound used, and the target insect species. At least an inactivating amount should be used. The term “insect-inactivating amount” is used to describe the amount, which is sufficient to cause a measurable reduction in the treated insect population. Generally an amount in the range from about 1 to about 1000 ppm by weight active compound is used. For example, insects or other pests which can be inhibited include, but are not limited to:

Lepidoptera—Heliothis spp., Helicoverpa spp., Spodoptera spp., Mythimna unipuncta, Agrotis ipsilon, Earias spp., Euxoa auxiliaris, Trichoplusia ni, Anticarsia gemmatalis, Rachiplusia nu, Plutella xylostella, Chilo spp., Scirpophaga incertulas, Sesamia inferens, Cnaphalocrocis medinalis, Ostrinia nubilalis, Cydia pomonella, Carposina niponensis, Adoxophyes orana, Archips argyrospilus, Pandemis heparana, Epinotia aporema, Eupoecilia ambiguella, Lobesia botrana, Polychrosis viteana, Pectinophora gossypiella, Pieris rapae, Phyllonorycter spp., Leucoptera malifoliella, Phyllocnisitis citrella

### Coleoptera—Diabrotica spp., Leptinotarsa decemlineata, Oulema oryzae, Anthonomus grandis, Lissorhoptrus oryzophilus, Agriotes spp., Melanotus communis, Popilliajaponica, Cyclocephala spp., Tribolium spp.

Homoptera—Aphis spp., Myzus persicae, Rhopalosiphum spp., Dysaphis plantaginea, Toxoptera spp., Macrosiphum euphorbiae, Aulacorthum solani, Sitobion avenae, Metopolophium dirhodum, Schizaphis graminum, Brachycolus noxius, Nephotettix spp., Nilaparvata lugens, Sogatella furcifera, Laodelphax striatellus, Bemisia tabaci, Trialeurodes vaporariorum, Aleurodes proletella, Aleurothrixus floccosus, Quadraspidiotus perniciosus, Unaspis yanonensis, Ceroplastes rubens, Aonidiella aurantii

### Hemiptera—Lygus spp., Eurygaster maura, Nezara viridula Piezodorus guildingi, Leptocorisa varicornis, Cimex lectularius, Cimex hemipterus

### Thysanoptera—Frankliniella spp., Thrips spp., Scirtothrips dorsalis

Isoptera—Reticulitermes flavipes, Coptotermes formosanus, Reticulitermes virginicus, Heterotermes aureus, Reticulitermes hesperus, Coptotermes frenchii, Shedorhinotennes spp., Reticulitermes santonensis, Reticulitermes grassei, Reticulitermes banyulensis, Reticulitermes speratus, Reticulitermes hageni, Reticulitermes tibialis, Zootermopsis spp., Incisitermes spp., Marginitermes spp., Macrotermes spp., Microcerotermes spp., Microtermes spp.

### Diptera—Liriomyza spp., Musca domestica, Aedes spp., Culex spp., Anopheles spp., Fannia spp., Stomoxys spp.,

### Hymenoptera—Iridomyrmex humilis, Solenopsis spp., Monomorium pharaonis, Atta spp., Pogonomyrmex spp., Camponotus spp., Monomorium spp., Tapinoma sessile, Tetramorium spp., Xylocapa spp., Vespula spp., Polistes spp.

### Mallophaga (chewing lice)

### Anoplura (sucking lice)—Pthirus pubis, Pediculus spp.

### Orthoptera (grasshoppers, crickets)—Melanoplus spp., Locusta migratoria, Schistocerca gregaria, Gryllotalpidae (mole crickets).

### Blattoidea (cockroaches)—Blatta orientalis, Blattella germanica, Periplaneta americana, Supella longipalpa, Periplaneta australasiae, Periplaneta brunnea, Parcoblatta pennsylvanica, Periplaneta fuliginosa, Pycnoscelus surinamensis,

### Siphonaptera—Ctenophalides spp., Pulex irritans

Acari—Tetranychus spp., Panonychus spp., Eotetranychus carpini, Phyllocoptruta oleivora, Aculus pelekassi, Brevipalpus phoenicis, Boophilus spp., Dermacentor variabilis, Rhipicephalus sanguineus, Amblyomma americanum, Ixodes spp., Notoedres cati, Sarcoptes scabiei, Dermatophagoides spp.

### Nematoda—Dirofilaria immitis, Meloidogyne spp., Heterodera spp., Hoplolaimus columbus, Belonolaimus spp., Pratylenchus spp., Rotylenchus reniformis, Criconemella ornata, Ditylenchus spp., Aphelenchoides besseyi, Hirschmanniella spp.

### Compositions

The compounds of this invention are applied in the form of compositions which are important embodiments of the invention, and which comprise a compound of this invention and a phytologically-acceptable inert carrier. Control of the pests is achieved by applying compounds of the invention in forms of sprays, topical treatment, gels, seed coatings, microcapsulations, systemic uptake, baits, eartags, boluses, foggers, fumigants aerosols, dusts and many others. The compositions are either concentrated solid or liquid formulations which are dispersed in water for application, or are dust or granular formulations which are applied without further treatment. The compositions are prepared according to procedures and formulae which are conventional in the agricultural chemical art, but which are novel and important because of the presence therein of the compounds of this invention. Some description of the formulation of the compositions will be given, however, to assure that agricultural chemists can readily prepare any desired composition.

The dispersions in which the compounds are applied are most often aqueous suspensions or emulsions prepared from concentrated formulations of the compounds. Such water-soluble, water-suspendable or emulsifiable formulations are either solids, usually known as wettable powders, or liquids usually known as emulsifiable concentrates or aqueous suspensions. Wettable powders, which may be compacted to form water dispersible granules, comprise an intimate mixture of the active compound, an inert carrier, and surfactants. The concentration of the active compound is usually from about 10% to about 90% by weight. The inert carrier is usually chosen from among the attapulgite clays, the montmorillonite clays, the diatomaceous earths, or the purified silicates. Effective surfactants, comprising from about 0.5% to about 10% of the wettable powder, are found among the sulfonated lignins, the condensed naphthalenesulfonates, the naphthalenesulfonates, the alkylbenzenesulfonates, the alkyl sulfates, and nonionic surfactants such as ethylene oxide adducts of alkyl phenols.

Emulsifiable concentrates of the compounds comprise a convenient concentration of a compound, such as from about 50 to about 500 grams per liter of liquid, equivalent to about 10% to about 50%, dissolved in an inert carrier which is either a water miscible solvent or a mixture of water-immiscible organic solvent and emulsifiers. Useful organic solvents include aromatics, especially the xylenes, and the petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are chosen from conventional anionic and/or nonionic surfactants, such as those discussed above.

Aqueous suspensions comprise suspensions of water-insoluble compounds of this invention, dispersed in an aqueous vehicle at a concentration in the range from about 5% to about 50% by weight. Suspensions are prepared by finely grinding the compound, and vigorously mixing it into a vehicle comprised of water and surfactants chosen from the same types discussed above. Inert ingredients, such as inorganic salts and synthetic or natural gums, may also be added, to increase the density and viscosity of the aqueous vehicle. It is often most effective to grind and mix the compound at the same time by preparing the aqueous mixture, and homogenizing it in an implement such as a sand mill, ball mill, or piston-type homogenizer.

The compounds may also be applied as granular compositions, which are particularly useful for applications to the soil. Granular compositions usually contain from about 0.5% to about 10% by weight of the compound, dispersed in an inert carrier which consists entirely or in large part of clay or a similar inexpensive substance. Such compositions are usually prepared by dissolving the compound in a suitable solvent and applying it to a granular carrier which has been pre-formed to the appropriate particle size, in the range of from about 0.5 to 3 mm. Such compositions may also be formulated by making a dough or paste of the carrier and compound and crushing and drying to obtain the desired granular particle size.

Dusts containing the compounds are prepared simply by intimately mixing the compound in powdered form with a suitable dusty agricultural carrier, such as kaolin clay, ground volcanic rock, and the like. Dusts can suitably contain from about 1% to about 10% of the compound.

It is equally practical, when desirable for any reason, to apply the compound in the form of a solution in an appropriate organic solvent, usually a bland petroleum oil, such as the spray oils, which are widely used in agricultural chemistry.

Insecticides and acaricides are generally applied in the form of a dispersion of the active ingredient in a liquid carrier. It is conventional to refer to application rates in terms of the concentration of active ingredient in the carrier. The most widely used carrier is water.

The compounds of the invention can also be applied in the form of an aerosol composition. In such compositions the active compound is dissolved or dispersed in an inert carrier, which is a pressure-generating propellant mixture. The aerosol composition is packaged in a container from which the mixture is dispensed through an atomizing valve. Propellant mixtures comprise either low-boiling halocarbons, which may be mixed with organic solvents, or aqueous suspensions pressurized with inert gases or gaseous hydrocarbons.

The actual amount of compound to be applied to loci of insects and mites is not critical and can readily be determined by those skilled in the art in view of the examples above. In general, concentrations from 10 ppm to 5000 ppm by weight of compound are expected to provide good control. With many of the compounds, concentrations from 100 to 1500 ppm will suffice.

The locus to which a compound is applied can be any locus inhabited by an insect or mite, for example, vegetable crops, fruit and nut trees, grape vines, ornamental plants, domesticated animals, the interior or exterior surfaces of buildings, and the soil around buildings.

Because of the unique ability of insect eggs to resist toxicant action, repeated applications may be desirable to control newly emerged larvae, as is true of other known insecticides and acaricides.

Systemic movement of compounds of the invention in plants may be utilized to control pests on one portion of the plant by applying the compounds to a different portion of it. For example, control of foliar-feeding insects can be controlled by drip irrigation or furrow application, or by treating the seed before planting. Seed treatment can be applied to all types of seeds, including those from which plants genetically transformed to express specialized traits will germinate. Representative examples include those expressing proteins toxic to invertebrate pests, such as Bacillus thuringiensis or other insecticidal proteins, those expressing herbicide resistance, such as “Roundup Ready®” seed, or those with “stacked” foreign genes expressing insecticidal proteins, herbicide resistance, nutrition-enhancement and/or any other beneficial traits.

An insecticidal bait composition consisting of compounds of the present invention and attractants and/or feeding stimulants may be used to increase efficacy of the insecticides against insect pest in a device such as trap, bait station, and the like. The bait composition is usually a solid, semi-solid (including gel) or liquid bait matrix including the stimulants and one or more non-microencapsulated or microencapsulated insecticides in an amount effective to act as kill agents.

The compounds of the present invention (Formula I) are often applied in conjunction with one or more other insecticides or fungicides or herbicides to obtain control of a wider variety of pests diseases and weeds. When used in conjunction with other insecticides or fungicides or herbicides, the presently claimed compounds can be formulated with the other insecticides or fungicides or herbicide, tank mixed with the other insecticides or fungicides or herbicides, or applied sequentially with the other insecticides or fungicides or herbicides.

Some of the insecticides that can be employed beneficially in combination with the compounds of the present invention include: antibiotic insecticides such as allosamidin and thuringiensin; macrocyclic lactone insecticides such as spinosad, spinetoram, and other spinosyns including the 21-butenyl spinosyns and their derivatives; avemectin insecticides such as abamectin, doramectin, emamectin, eprinomectin, ivermectin and selamectin; milbemycin insecticides such as lepimectin, milbemectin, milbemycin oxime and moxidectin; arsenical insecticides such as calcium arsenate, copper acetoarsenite, copper arsenate, lead arsenate, potassium arsenite and sodium arsenite; biological insecticides such as Bacillus popilliae, B. sphaericus, B. thuringiensis subsp. aizawai, B. thuringiensis subsp. kurstaki, B. thuringiensis subsp. tenebrionis, Beauveria bassiana, Cydia pomonella granulosis virus, Douglas fir tussock moth NPV, gypsy moth NPV, Helicoverpa zea NPV, Indian meal moth granulosis virus, Metarhizium anisopliae, Nosema locustae, Paecilomyces fumosoroseus, P. lilacinus, Photorhabdus luminescens, Spodoptera exigua NPV, trypsin modulating oostatic factor, Xenorhabdus nematophilus, and X. bovienii, plant incorporated protectant insecticides such as Cry1Ab, Cry1Ac, Cry1F, Cry1A.105, Cry2Ab2, Cry3A, mir Cry3A, Cry3Bb1, Cry34, Cry35, and VIP3A; botanical insecticides such as anabasine, azadirachtin, d-limonene, nicotine, pyrethrins, cinerins, cinerin I, cinerin II, jasmolin I, jasmolin II, pyrethrin I, pyrethrin II, quassia, rotenone, ryania and sabadilla; carbamate insecticides such as bendiocarb and carbaryl; benzofuranyl methylcarbamate insecticides such as benfuracarb, carbofuran, carbosulfan, decarbofuran and furathiocarb; dimethylcarbamate insecticides dimitan, dimetilan, hyquincarb and pirimicarb; oxime carbamate insecticides such as alanycarb, aldicarb, aldoxycarb, butocarboxim, butoxycarboxim, methomyl, nitrilacarb, oxamyl, tazimcarb, thiocarboxime, thiodicarb and thiofanox; phenyl methylcarbamate insecticides such as allyxycarb, aminocarb, bufencarb, butacarb, carbanolate, cloethocarb, dicresyl, dioxacarb, EMPC, ethiofencarb, fenethacarb, fenobucarb, isoprocarb, methiocarb, metolcarb, mexacarbate, promacyl, promecarb, propoxur, trimethacarb, XMC and xylylcarb; dinitrophenol insecticides such as dinex, dinoprop, dinosam and DNOC; fluorine insecticides such as barium hexafluorosilicate, cryolite, sodium fluoride, sodium hexafluorosilicate and sulfluramid; formamidine insecticides such as amitraz, chlordimeform, formetanate and formparanate; fumigant insecticides such as acrylonitrile, carbon disulfide, carbon tetrachloride, chloroform, chloropicrin, para-dichlorobenzene, 1,2-dichloropropane, ethyl formate, ethylene dibromide, ethylene dichloride, ethylene oxide, hydrogen cyanide, iodomethane, methyl bromide, methylchloroform, methylene chloride, naphthalene, phosphine, sulfuryl fluoride and tetrachloroethane; inorganic insecticides such as borax, calcium polysulfide, copper oleate, mercurous chloride, potassium thiocyanate and sodium thiocyanate; chitin synthesis inhibitors such as bistrifluron, buprofezin, chlorfluazuron, cyromazine, diflubenzuron, flucycloxuron, flufenoxuron, hexaflumuron, lufenuron, novaluron, noviflumuron, penfluron, teflubenzuron and triflumuron; juvenile hormone mimics such as epofenonane, fenoxycarb, hydroprene, kinoprene, methoprene, pyriproxyfen and triprene; juvenile hormones such as juvenile hormone I, juvenile hormone II and juvenile hormone III; moulting hormone agonists such as chromafenozide, halofenozide, methoxyfenozide and tebufenozide; moulting hormones such as α-ecdysone and ecdysterone; moulting inhibitors such as diofenolan; precocenes such as precocene I, precocene II and precocene III; unclassified insect growth regulators such as dicyclanil; nereistoxin analogue insecticides such as bensultap, cartap, thiocyclam and thiosultap; nicotinoid insecticides such as flonicamid; nitroguanidine insecticides such as clothianidin, dinotefuran, imidacloprid and thiamethoxam; nitromethylene insecticides such as nitenpyram and nithiazine; pyridylmethylamine insecticides such as acetamiprid, imidacloprid, nitenpyram and thiacloprid; organochlorine insecticides such as bromo-DDT, camphechlor, DDT, pp′-DDT, ethyl-DDD, HCH, gamma-HCH, lindane, methoxychlor, pentachlorophenol and TDE; cyclodiene insecticides such as aldrin, bromocyclen, chlorbicyclen, chlordane, chlordecone, dieldrin, dilor, endosulfan, endrin, HEOD, heptachlor, HHDN, isobenzan, isodrin, kelevan and mirex; organophosphate insecticides such as bromfenvinfos, chlorfenvinphos, crotoxyphos, dichlorvos, dicrotophos, dimethylvinphos, fospirate, heptenophos, methocrotophos, mevinphos, monocrotophos, naled, naftalofos, phosphamidon, propaphos, TEPP and tetrachlorvinphos; organothiophosphate insecticides such as dioxabenzofos, fosmethilan and phenthoate; aliphatic organothiophosphate insecticides such as acethion, amiton, cadusafos, chlorethoxyfos, chlormephos, demephion, demephion-O, demephion-S, demeton, demeton-O, demeton-S, demeton-methyl, demeton-O-methyl, demeton-S-methyl, demeton-S-methylsulphon, disulfoton, ethion, ethoprophos, IPSP, isothioate, malathion, methacrifos, oxydemeton-methyl, oxydeprofos, oxydisulfoton, phorate, sulfotep, terbufos and thiometon; aliphatic amide organothiophosphate insecticides such as amidithion, cyanthoate, dimethoate, ethoate-methyl, formothion, mecarbam, omethoate, prothoate, sophamide and vamidothion; oxime organothiophosphate insecticides such as chlorphoxim, phoxim and phoxim-methyl; heterocyclic organothiophosphate insecticides such as azamethiphos, coumaphos, coumithoate, dioxathion, endothion, menazon, morphothion, phosalone, pyraclofos, pyridaphenthion and quinothion; benzothiopyran organothiophosphate insecticides such as dithicrofos and thicrofos; benzotriazine organothiophosphate insecticides such as azinphos-ethyl and azinphos-methyl; isoindole organothiophosphate insecticides such as dialifos and phosmet; isoxazole organothiophosphate insecticides such as isoxathion and zolaprofos; pyrazolopyrimidine organothiophosphate insecticides such as chlorprazophos and pyrazophos; pyridine organothiophosphate insecticides such as chlorpyrifos and chlorpyrifos-methyl; pyrimidine organothiophosphate insecticides such as butathiofos, diazinon, etrimfos, lirimfos, pirimiphos-ethyl, pirimiphos-methyl, primidophos, pyrimitate and tebupirimfos; quinoxaline organothiophosphate insecticides such as quinalphos and quinalphos-methyl; thiadiazole organothiophosphate insecticides such as athidathion, lythidathion, methidathion and prothidathion; triazole organothiophosphate insecticides such as isazofos and triazophos; phenyl organothiophosphate insecticides such as azothoate, bromophos, bromophos-ethyl, carbophenothion, chlorthiophos, cyanophos, cythioate, dicapthon, dichlofenthion, etaphos, famphur, fenchlorphos, fenitrothion fensulfothion, fenthion, fenthion-ethyl, heterophos, jodfenphos, mesulfenfos, parathion, parathion-methyl, phenkapton, phosnichlor, profenofos, prothiofos, sulprofos, temephos, trichlormetaphos-3 and trifenofos; phosphonate insecticides such as butonate and trichlorfon; phosphonothioate insecticides such as mecarphon; phenyl ethylphosphonothioate insecticides such as fonofos and trichloronat; phenyl phenylphosphonothioate insecticides such as cyanofenphos, EPN and leptophos; phosphoramidate insecticides such as crufomate, fenamiphos, fosthietan, mephosfolan, phosfolan and pirimetaphos; phosphoramidothioate insecticides such as acephate, isocarbophos, isofenphos, methamidophos and propetamphos; phosphorodiamide insecticides such as dimefox, mazidox, mipafox and schradan; oxadiazine insecticides such as indoxacarb; phthalimide insecticides such as dialifos, phosmet and tetramethrin; pyrazole insecticides such as acetoprole, ethiprole, fipronil, pyrafluprole, pyriprole, tebufenpyrad, tolfenpyrad and vaniliprole; pyrethroid ester insecticides such as acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin and transfluthrin; pyrethroid ether insecticides such as etofenprox, flufenprox, halfenprox, protrifenbute and silafluofen; pyrimidinamine insecticides such as flufenerim and pyrimidifen; pyrrole insecticides such as chlorfenapyr; tetronic acid insecticides such as spirodiclofen, spiromesifen and spirotetramat; thiourea insecticides such as diafenthiuron; urea insecticides such as flucofuron and sulcofuron; and unclassified insecticides such as AKD-3088, closantel, crotamiton, cyflumetofen, E2Y45, EXD, fenazaflor, fenazaquin, fenoxacrim, fenpyroximate, FKI-1033, flubendiamide, HGW86, hydramethylnon, IKI-2002, isoprothiolane, malonoben, metaflumizone, metoxadiazone, nifluridide, NNI-9850, NNI-0101, pymetrozine, pyridaben, pyridalyl, Qcide, rafoxanide, rynaxypyr, SYJ-159, triarathene and triazamate and any combinations thereof.

Some of the fungicides that can be employed beneficially in combination with the compounds of the present invention include: 2-(thiocyanatomethylthio)-benzothiazole, 2-phenylphenol, 8-hydroxyquinoline sulfate, Ampelomyces, quisqualis, azaconazole, azoxystrobin, Bacillus subtilis, benalaxyl, benomyl, benthiavalicarb-isopropyl, benzylaminobenzene-sulfonate (BABS) salt, bicarbonates, biphenyl, bismerthiazol, bitertanol, blasticidin-S, borax, Bordeaux mixture, boscalid, bromuconazole, bupirimate, calcium polysulfide, captafol, captan, carbendazim, carboxin, carpropamid, carvone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fentin, fentin acetate, fentin hydroxide, ferbam, ferimzone, fluazinam, fludioxonil, flumorph, fluopicolide, fluoroimide, fluoxastrobin, fluquinconazole, flusilazole, flusulfamide, flutolanil, flutriafol, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), ipconazole, iprobenfos, iprodione, iprovalicarb, isoprothiolane, kasugamycin, kasugamycin hydrochloride hydrate, kresoxim-methyl, mancopper, mancozeb, maneb, mepanipyrim, mepronil, mercuric chloride, mercuric oxide, mercurous chloride, metalaxyl, mefenoxam, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxine-copper, oxpoconazole fumarate, oxycarboxin, pefurazoate, penconazole, pencycuron, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pyraclostrobin, pyrazophos, pyributicarb, pyrifenox, pyrimethanil, pyroquilon, quinoclamine, quinoxyfen, quintozene, Reynoutria sachalinensis extract, silthiofam, simeconazole, sodium 2-phenylphenoxide, sodium bicarbonate, sodium pentachlorophenoxide, spiroxamine, sulfur, SYP-Z071, tar oils, tebuconazole, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, vinclozolin, zineb, ziram, zoxamide, Candida oleophila, Fusarium oxysporum, Gliocladium spp., Phlebiopsis gigantean, Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme: ampropylfos, anilazine, azithiram, barium polysulfide, Bayer 32394, benodanil, benquinox, bentaluron, benzamacril; benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, cufraneb, cupric hydrazinium sulfate, cuprobam, cyclafuramid, cypendazole, cyprofuram, decafentin, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, fluotrimazole, furcarbanil, furconazole, furconazole-cis, furmecyclox, furophanate, glyodine, griseofulvin, halacrinate, Hercules 3944, hexylthiofos, ICIA0858, isopamphos, isovaledione, mebenil, mecarbinzid, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, phenylmercury dimethyldithiocarbamate, phenylmercury nitrate, phosdiphen, prothiocarb; prothiocarb hydrochloride, pyracarbolid, pyridinitril, pyroxychlor, pyroxyfur, quinacetol; quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, urbacid, XRD-563, and zarilamid, and any combinations thereof.

Some of the herbicides that can be employed in conjunction with the compounds of the present invention include: amide herbicides such as allidochlor, beflubutamid, benzadox, benzipram, bromobutide, cafenstrole, CDEA, chlorthiamid, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid and tebutam; anilide herbicides such as chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen and propanil; arylalanine herbicides such as benzoylprop, flamprop and flamprop-M; chloroacetanilide herbicides such as acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor and xylachlor; sulfonanilide herbicides such as benzofluor, perfluidone, pyrimisulfan and profluazol; sulfonamide herbicides such as asulam, carbasulam, fenasulam and oryzalin; antibiotic herbicides such as bilanafos; benzoic acid herbicides such as chloramben, dicamba, 2,3,6-TBA and tricamba; pyrimidinyloxybenzoic acid herbicides such as bispyribac and pyriminobac; pyrimidinylthiobenzoic acid herbicides such as pyrithiobac; phthalic acid herbicides such as chlorthal; picolinic acid herbicides such as aminopyralid, clopyralid and picloram; quinolinecarboxylic acid herbicides such as quinclorac and quinmerac; arsenical herbicides such as cacodylic acid, CMA, DSMA, hexaflurate, MAA, MAMA, MSMA, potassium arsenite and sodium arsenite; benzoylcyclohexanedione herbicides such as mesotrione, sulcotrione, tefuryltrione and tembotrione; benzofuranyl alkylsulfonate herbicides such as benfuresate and ethofumesate; carbamate herbicides such as asulam, carboxazole chlorprocarb, dichlormate, fenasulam, karbutilate and terbucarb; carbanilate herbicides such as barban, BCPC, carbasulam, carbetamide, CEPC, chlorbufam, chlorpropham, CPPC, desmedipham, phenisopham, phenmedipham, phenmedipham-ethyl, propham and swep; cyclohexene oxime herbicides such as alloxydim, butroxydim, clethodim, cloproxydim, cycloxydim, profoxydim, sethoxydim, tepraloxydim and tralkoxydim; cyclopropylisoxazole herbicides such as isoxachlortole and isoxaflutole; dicarboximide herbicides such as benzfendizone, cinidon-ethyl, flumezin, flumiclorac, flumioxazin and flumipropyn; dinitroaniline herbicides such as benfluralin, butralin, dinitramine, ethalfluralin, fluchloralin, isopropalin, methalpropalin, nitralin, oryzalin, pendimethalin, prodiamine, profluralin and trifluralin; dinitrophenol herbicides such as dinofenate, dinoprop, dinosam, dinoseb, dinoterb, DNOC, etinofen and medinoterb; diphenyl ether herbicides such as ethoxyfen; nitrophenyl ether herbicides such as acifluorfen, aclonifen, bifenox, chlomethoxyfen, chlornitrofen, etnipromid, fluorodifen, fluoroglycofen, fluoronitrofen, fomesafen, furyloxyfen, halosafen, lactofen, nitrofen, nitrofluorfen and oxyfluorfen; dithiocarbamate herbicides such as dazomet and metam; halogenated aliphatic herbicides such as alorac, chloropon, dalapon, flupropanate, hexachloroacetone, iodomethane, methyl bromide, monochloroacetic acid, SMA and TCA; imidazolinone herbicides such as imazamethabenz, imazamox, imazapic, imazapyr, imazaquin and imazethapyr; inorganic herbicides such as ammonium sulfamate, borax, calcium chlorate, copper sulfate, ferrous sulfate, potassium azide, potassium cyanate, sodium azide, sodium chlorate and sulfuric acid; nitrile herbicides such as bromobonil, bromoxynil, chloroxynil, dichlobenil, iodobonil, ioxynil and pyraclonil; organophosphorus herbicides such as amiprofos-methyl, anilofos, bensulide, bilanafos, butamifos, 2,4-DEP, DMPA, EBEP, fosamine, glufosinate, glyphosate and piperophos; phenoxy herbicides such as bromofenoxim, clomeprop, 2,4-DEB, 2,4-DEP, difenopenten, disul, erbon, etnipromid, fenteracol and trifopsime; phenoxyacetic herbicides such as 4-CPA, 2,4-D, 3,4-DA, MCPA, MCPA-thioethyl and 2,4,5-T; phenoxybutyric herbicides such as 4-CPB, 2,4-DB, 3,4-DB, MCPB and 2,4,5-TB; phenoxypropionic herbicides such as cloprop, 4-CPP, dichlorprop, dichlorprop-P, 3,4-DP, fenoprop, mecoprop and mecoprop-P; aryloxyphenoxypropionic herbicides such as chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P and trifop; phenylenediamine herbicides such as dinitramine and prodiamine; pyrazolyl herbicides such as benzofenap, pyrazolynate, pyrasulfotole, pyrazoxyfen, pyroxasulfone and topramezone; pyrazolylphenyl herbicides such as fluazolate and pyraflufen; pyridazine herbicides such as credazine, pyridafol and pyridate; pyridazinone herbicides such as brompyrazon, chloridazon, dimidazon, flufenpyr, metflurazon, norflurazon, oxapyrazon and pydanon; pyridine herbicides such as aminopyralid, cliodinate, clopyralid, dithiopyr, fluroxypyr, haloxydine, picloram, picolinafen, pyriclor, thiazopyr and triclopyr; pyrimidinediamine herbicides such as iprymidam and tioclorim; quaternary ammonium herbicides such as cyperquat, diethamquat, difenzoquat, diquat, morfamquat and paraquat; thiocarbamate herbicides such as butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate and vemolate; thiocarbonate herbicides such as dimexano, EXD and proxan; thiourea herbicides such as methiuron; triazine herbicides such as dipropetryn, triaziflam and trihydroxytriazine; chlorotriazine herbicides such as atrazine, chlorazine, cyanazine, cyprazine, eglinazine, ipazine, mesoprazine, procyazine, proglinazine, propazine, sebuthylazine, simazine, terbuthylazine and trietazine; methoxytriazine herbicides such as atraton, methometon, prometon, secbumeton, simeton and terbumeton; methylthiotriazine herbicides such as ametryn, aziprotryne, cyanatryn, desmetryn, dimethametryn, methoprotryne, prometryn, simetryn and terbutryn; triazinone herbicides such as ametridione, amibuzin, hexazinone, isomethiozin, metamitron and metribuzin; triazole herbicides such as amitrole, cafenstrole, epronaz and flupoxam; triazolone herbicides such as amicarbazone, bencarbazone, carfentrazone, flucarbazone, propoxycarbazone, sulfentrazone and thiencarbazone-methyl; triazolopyrimidine herbicides such as cloransulam, diclosulam, florasulam, flumetsulam, metosulam, penoxsulam and pyroxsulam; uracil herbicides such as butafenacil, bromacil, flupropacil, isocil, lenacil and terbacil; 3-phenyluracils; urea herbicides such as benzthiazuron, cumyluron, cycluron, dichloralurea, diflufenzopyr, isonoruron, isouron, methabenzthiazuron, monisouron and noruron; phenylurea herbicides such as anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluron, phenobenzuron, siduron, tetrafluron and thidiazuron; pyrimidinylsulfonylurea herbicides such as amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron and trifloxysulfuron; triazinylsulfonylurea herbicides such as chlorsulfuron, cinosulfuron, ethametsulfuron, iodosulfuron, metsulfuron, prosulfuron, thifensulfuron, triasulfuron, tribenuron, triflusulfuron and tritosulfuron; thiadiazolylurea herbicides such as buthiuron, ethidimuron, tebuthiuron, thiazafluron and thidiazuron; and unclassified herbicides such as acrolein, allyl alcohol, azafenidin, benazolin, bentazone, benzobicyclon, buthidazole, calcium cyanamide, cambendichlor, chlorfenac, chlorfenprop, chlorflurazole, chlorflurenol, cinmethylin, clomazone, CPMF, cresol, ortho-dichlorobenzene, dimepiperate, endothal, fluoromidine, fluridone, flurochloridone, flurtamone, fluthiacet, indanofan, methazole, methyl isothiocyanate, nipyraclofen, OCH, oxadiargyl, oxadiazon, oxaziclomefone, pentachlorophenol, pentoxazone, phenylmercury acetate, pinoxaden, prosulfalin, pyribenzoxim, pyriftalid, quinoclamine, rhodethanil, sulglycapin, thidiazimin, tridiphane, trimeturon, tripropindan and tritac.

We claim:

1. Methods to control certain invertebrates including insects in agricultural, urban, animal health, and industrial systems by directly or systemically applying to a locus where control is desired an effective amount of a compound of formula (I) 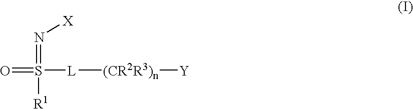
wherein
X represents NO₂, CN or COOR⁴;
L represents a single bond or —CH— (CH₂)ₘ— wherein m is an integer from 1-3 in cases where R¹, S and L taken together represent a 4-, 5- or 6-membered ring;
R¹ represents methyl, ethyl, or —CH₂— in cases where R¹, S and L taken together represent a 4-, 5- or 6-membered ring;
R² and R³ independently represent hydrogen, methyl, ethyl, fluoro, chloro or bromo;
n is an integer from 0-3;
Y represents 6-halopyridin-3-yl, 6-(C₁-C₄)alkylpyridin-3-yl, 6-(C₁-C₄)alkoxypyridin-3-yl, 2-chlorothiazol-4-yl, 2-chlorothiazol-5-yl, or 3-chloroisoxazol-5-yl; and
R⁴ represents C₁-C₃ alkyl.

2. The method of claim 1 in which X in the compound of Formula I represents CN.

3. The method of claim 1 in which the compound of Formula I has the structure 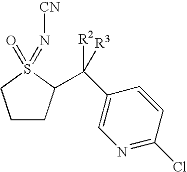
wherein
R² and R³ independently represent hydrogen or methyl.

4. The method of claim 1 in which the compound of Formula I has the structure 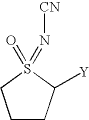
wherein
Y represents 6-halopyridin-3-yl.

5. The method of claim 1 in which the compound of Formula I has the structure 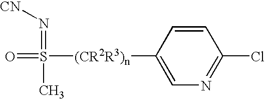
wherein
R² and R³ independently represent hydrogen or methyl; and
n=1-3.

6. The method of claim 1 in which a compound of formula I is used to control insects systemically by root uptake.

7. The method of claim 1 in which a compound of formula I is used to control insects systemically by seed treatment.

8. The method of claim 1 in which a compound of formula I is used to control termites by contact or ingestion.

9. The method of claim 1 in which a compound of formula I is used to control fleas by contact or ingestion.

10. The method of claim 1 in which a compound of formula I is used to control ticks by contact or ingestion.
